(19) United States
(12) Patent Application Publication
(10) Pub. No.: US 2026/0158099 A1
(43) Pub. Date: Jun. 11, 2026

(54) METHOD FOR TREATING STOMACH DISORDERS

(71) Applicant: Zhang Hong Fang, Ottawa (CA)

(72) Inventor: Zhang Hong Fang, Ottawa (CA)

(21) Appl. No.: 18/976,607

(22) Filed: Dec. 11, 2024

(51) Int. Cl. A61K 36/899

(52) U.S. Cl. CPC A61K 36/899

(57) ABSTRACT

A method for treating stomach maladies wherein the method includes preparation of a proprietary formula and an administration thereof. The method utilizes whole wheat flour wherein the whole wheat flour is cooked until the color of the whole wheat flour is brown. The formulation of the method further includes utilization of millet flour wherein the millet flour is cooked until the color thereof is brown. The cooked flours are combined wherein the ratio thereof is two-thirds whole wheat flour to one-third millet flour in the final volume of the combined flour. The flour mixture is added to boiling water wherein approximately four tablespoons of the flour mixture is added to one cup of water. The solution is administered orally to the user wherein the solution is administered at a temperature of fifty degrees Celsius. Brown sugar can further be included into the dissolved flour mixture.

### FIELD OF THE INVENTION

The present invention relates generally to methods for treating illnesses and discomfort, more specifically but not by way of limitation, a method of treating stomach disorders wherein the method of the present invention includes preparation of a formulation and administration thereof.

### BACKGROUND

As is known in the art, the stomach contains several muscles. The stomach can change shape during eating or during a posture change. The stomach plays an instrumental role in digestion. When an individual swallows, food travels down the esophagus, passes the lower esophageal sphincter, and enters the stomach. The stomach has three primary functions. First, the stomach provides temporary storage of food and liquids. The stomach also is the organ responsible for production of digestive juices. Lastly, the stomach will facilitate the transfer of the food and liquid mixture into the small intestine. How long this process takes depends on the foods eaten and how well the stomach muscles function. Certain foods, like carbohydrates, pass through quickly, while proteins remain longer. Fats take the most time to process.

Stomach diseases and disorders are one of the most common maladies affecting people in most countries. One stomach malady is commonly known as reflux. Reflux occurs when stomach contents such as food, acid, or bile moves back into your esophagus. When this happens twice a week or more, it's called gastroesophageal reflux disease. This chronic condition can cause heartburn and irritate your esophagus lining. Another stomach malady is gastritis. Gastritis is an inflammation of the stomach lining. According to some research studies, 8 in 1,000 people have acute gastritis and 2 of every 10,000 develop chronic gastritis. If the lining of the stomach breaks down a peptic ulcer can develop. Most peptic ulcers are located in the first layer of the inner lining. An ulcer that goes all the way through your stomach lining is called a perforation and requires immediate medical attention Viral gastroenteritis occurs when a virus causes the stomach and intestines to become inflamed. The main symptoms are vomiting and diarrhea. Most people recover within a few days. Very young children, older adults, and people with other diseases are at increased risk for dehydration. While over the counter and prescription treatments are available to treat the aforementioned stomach maladies, these medications have long term side effects and as such a safer alternative is needed.

Accordingly, there is a need for a method of producing and administering a formulation that is operable to safely treat a plurality of stomach maladies.

### SUMMARY OF THE INVENTION

It is the object of the present invention to provide a method of preparing and administering a formulation for treatment of stomach maladies wherein the present invention utilizes whole wheat flour and millet flour in the formulation.

Another object of the present invention is to provide a method for treating stomach disorders wherein the method of the present invention employs a ratio of whole wheat flour to millet flour of two-thirds whole wheat and one-third millet flour.

A further object of the present invention is to provide a method of preparing and administering a formulation for treatment of stomach maladies wherein the present invention includes a step of heating the whole wheat flour and millet flour wherein during the heating process the color thereof changes to brown.

Yet a further object of the present invention is to provide a method for treating stomach disorders wherein the method of the present invention further utilizes brown sugar in the formulation.

Still another object of the present invention is to provide a method of preparing and administering a formulation for treatment of stomach maladies wherein the method of the administration includes utilization of boiling water to which the whole wheat flour, millet flour and brown sugar is added.

An additional object of the present invention is to provide a method for treating stomach disorders wherein the method of the present invention includes administration of the formulation of the present invention wherein the formulation is at a temperature of approximately fifty degrees Celsius.

Yet a further object of the present invention is to provide a method of preparing and administering a formulation for treatment of stomach maladies wherein the present invention includes a step of administering the formulation fifteen minutes prior to eating a meal.

To the accomplishment of the above and related objects the present invention may be embodied in the form illustrated in the accompanying drawings. Attention is called to the fact that the drawings are illustrative only. Variations are contemplated as being a part of the present invention, limited only by the scope of the claims.

### BRIEF DESCRIPTION OF THE DRAWINGS

A more complete understanding of the present invention may be had by reference to the following Detailed Description and appended claims when taken in conjunction with the accompanying Drawings wherein:

FIG. 1 is a flow chart of the process of preparation and administering of the present invention.

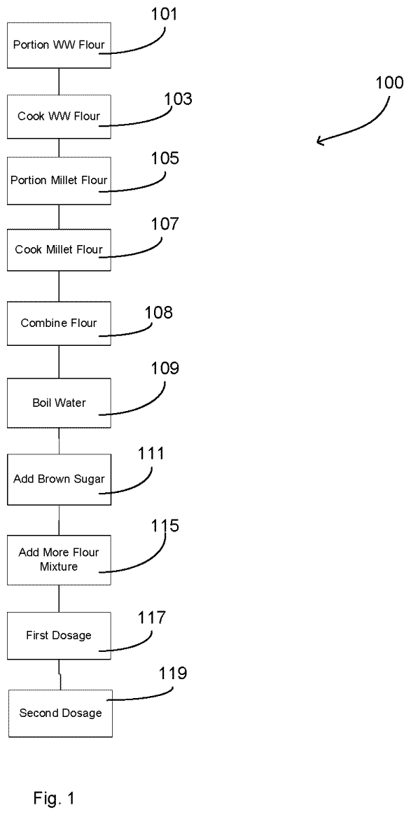

### DETAILED DESCRIPTION

Referring now to the drawings submitted herewith, wherein various elements depicted therein are not necessarily drawn to scale and wherein through the views and figures like elements are referenced with identical reference numerals, there is illustrated a method for treating stomach maladies 100 constructed according to the principles of the present invention.

An embodiment of the present invention is discussed herein with reference to the figures submitted herewith. Those skilled in the art will understand that the detailed description herein with respect to these figures is for explanatory purposes and that it is contemplated within the scope of the present invention that alternative embodiments are plausible. By way of example but not by way of limitation, those having skill in the art in light of the present teachings of the present invention will recognize a plurality of alternate and suitable approaches dependent upon the needs of the particular application to implement the functionality of any given detail described herein, beyond that of the particular implementation choices in the embodiment described herein. Various modifications and embodiments are within the scope of the present invention.

It is to be further understood that the present invention is not limited to the particular methodology, materials, uses and applications described herein, as these may vary. Furthermore, it is also to be understood that the terminology used herein is used for the purpose of describing particular embodiments only, and is not intended to limit the scope of the present invention. It must be noted that as used herein and in the claims, the singular forms “a”, “an” and “the” include the plural reference unless the context clearly dictates otherwise. Thus, for example, a reference to “an element” is a reference to one or more elements and includes equivalents thereof known to those skilled in the art. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

References to “one embodiment”, “an embodiment”, “exemplary embodiments”, and the like may indicate that the embodiment(s) of the invention so described may include a particular feature, structure or characteristic, but not every embodiment necessarily includes the particular feature, structure or characteristic.

Referring in particular to the Figures submitted herewith, the method for treating stomach maladies 100 is discussed herein in its preferred embodiment. It should be understood within the scope of the present invention that variations of parameters such as but not limited to flour portioning are contemplated within the scope of the present invention. In step 101, the user of the method for treating stomach maladies 100 will portion an amount of whole wheat flour from a source utilizing suitable techniques and implements. Step 103, the user will cook the whole wheat flour wherein during the cooking process the whole wheat flour is cooked until the color of the whole wheat flour is brown. In step 105, the user of the method for treating stomach maladies 100 will portion an amount of millet flour from a source utilizing suitable techniques and implements. Step 107, the user cooks the millet flour utilizing a suitable cooking utensil wherein the millet flour is cooked until the millet flour is brown in color. In step 108, the user will combine the whole wheat flour and the millet flour, both having been cooked to a brown color, wherein the whole wheat flour is combined at a ratio with the millet flour of two-thirds whole wheat flour to one third millet flour. It should be understood within the scope of the present invention that the total volume of the flour mixture could vary but that ratio of two-thirds whole wheat flour to one third millet flour must be achieved.

Step 109, the user of the method for treating stomach maladies 100 obtains water from a desired source and places in an implement suitable for boiling thereof and commences boiling the water. Step 111, a desired amount of brown sugar is added to the boiling water. It should be understood that the brown sugar could be eliminated or added to the water at an amount of two tablespoons. It should be further understood within the scope of the present invention that the water volume utilized in the aforementioned process is eight fluid ounces of water. In step 115, the user of the method for treating stomach maladies 100 adds the flour mixture to the boiling water wherein the user will add approximately four tablespoons of the flour mixture to the boiling water for dissolving thereof. Step 117, the user will administer the first dosage of the formulation wherein the first dosage is administered to the user in the morning approximately fifteen minutes prior to eating any food. In step 119, the second dosage is administered to the user wherein the second dosage is delivered fifteen minutes prior to the last meal of the day for the user. It should be understood within the scope of the present invention that the first dosage and second dosage are administered at a temperature of fifty degrees Celsius.

While a daily administration of two doses are described herein, it should be understood within the scope of the present invention that the formulation of the present invention can be administered at alternate times and reactively to treat symptoms such as stomach pains.

In the preceding detailed description, reference has been made to the accompanying drawings that form a part hereof, and in which are shown by way of illustration specific embodiments in which the invention may be practiced. These embodiments, and certain variants thereof, have been described in sufficient detail to enable those skilled in the art to practice the invention. It is to be understood that other suitable embodiments may be utilized and that logical changes may be made without departing from the spirit or scope of the invention. The description may omit certain information known to those skilled in the art. The preceding detailed description is, therefore, not intended to be limited to the specific forms set forth herein, but on the contrary, it is intended to cover such alternatives, modifications, and equivalents, as can be reasonably included within the spirit and scope of the appended claims.

What is claimed is:

1. A method for treating stomach maladies wherein the method comprises the steps of:
obtaining a portion of whole wheat flour, wherein the whole wheat flour is portioned from a desired source;
cooking the portion of the whole wheat flour, wherein the whole wheat flour is cooked until a color thereof is brown;
obtaining a portion of millet flour, wherein the millet flour is portioned from a desired source;
cooking the portion of the millet flour, wherein the millet flour is cooked until a color thereof is brown;
combining the whole wheat flour and the millet flour to create a flour mixture, wherein the flour mixture has a ratio of whole wheat flour to millet flour;
boiling a volume of water;
adding an amount of the flour mixture to the boiling water;
dissolving the flour mixture in the boiling water to create an ingestible formulation;
administering a first dose of the formulation, wherein the administration of the first dose of the formulation is performed at a first time; and
administering a second dose of the formulation, wherein the administration of the second dose of the formulation is performed at a second time.

2. The method for treating stomach maladies as recited in claim 1, and further including adding an amount of brown sugar to the formulation.

3. The method for treating stomach maladies as recited in claim 2, wherein the flour mixture has a ratio of two-thirds whole wheat flour to one-third millet flour.

4. The method for treating stomach maladies as recited in claim 3, wherein the first time is fifteen minutes prior to a first meal of a day for a user.

5. The method for treating stomach maladies as recited in claim 4, wherein the formulation is provided to a user at a temperature of fifty degrees Celsius.

6. The method for treating stomach maladies as recited in claim 5, wherein the second time is fifteen minutes prior to a last meal of a day for a user.
